(19) United States
(12) Patent Application Publication
(10) Pub. No.: US 2022/0280053 A1
(43) Pub. Date: Sep. 8, 2022

(54) ELECTRONIC APPARATUS, AND METHOD

(71) Applicant: KABUSHIKI KAISHA TOSHIBA, Tokyo (JP)

(72) Inventor: Ken KAWAKAMI, Kawasaki Kanagawa (JP)

(73) Assignee: KABUSHIKI KAISHA TOSHIBA, Tokyo (JP)

(21) Appl. No.: 17/470,486

(22) Filed: Sep. 9, 2021

(30) Foreign Application Priority Data
Mar. 8, 2021 (JP) 2021-036611

(51) Int. Cl. A61B 5/021; A61B 5/022; A61B 5/026; A61B 5/00

(52) U.S. Cl. CPC A61B 5/02125; A61B 5/022; A61B 5/026; A61B 5/02116; A61B 5/7239; A61B 2560/0223

(57) ABSTRACT

According to the present embodiment, an electronic apparatus comprising a processor configured to generate a calibration period corresponding to a pressurization period of a pressurizer for a subject using a received light signal based on scattered light scattered in a body of the subject when an optical signal of a predetermined frequency band is applied thereto, acquire a first blood pressure based on the received light signal in the calibration period, and to generate calibration information using a reference blood pressure measured on a basis of pressurization of the pressurizer, and the first blood pressure.

### CROSS-REFERENCE TO RELATED APPLICATIONS

This application is based upon and claims the benefit of priority from the prior Japanese Patent Application No. 2021-036611, filed on Mar. 8, 2021 the entire contents of which are incorporated herein by reference.

### FIELD

Embodiments of the present invention relate to an electronic apparatus, and a method.

### BACKGROUND

A PPG (Photoplethysmogram) sensor that detects a pulse wave associated with heartbeat by measuring changes of the blood volume in arteries and capillaries corresponding to changes of the heart rate is known. A method of detecting the heart rate on the basis of the volume of blood passing through tissues with respect to each beat using a PPG sensor is called BVP (Blood Volume Pulse) measurement.

However, the sensitivity of the PPG sensor is likely to differ according to manufacturers or to be influenced by measurement environments.

### BRIEF DESCRIPTION OF THE DRAWINGS

FIG. 1 is a block diagram illustrating a schematic configuration of a blood pressure processing apparatus according to the present embodiment;

FIG. 2 is a block diagram illustrating a configuration example of a measuring part;

FIG. 3 is a block diagram illustrating a configuration example of a calibration processor;

FIG. 4 is a diagram illustrating an example of a measurement signal of a subject A;

FIG. 5 is a diagram illustrating an example of a volume pulse wave;

FIG. 6 is a diagram illustrating an example of the measurement signal of a subject B;

FIG. 7 is a diagram illustrating an example of the measurement signal of a subject C;

FIG. 8 is a diagram illustrating an example of the measurement signal of a subject D;

FIG. 9 is a diagram illustrating an example of the measurement signal during a calibration period;

FIG. 10 is a diagram illustrating a relation between a first blood pressure in the calibration period and a reference blood pressure;

FIG. 11 is a block diagram illustrating a configuration example of a blood pressure acquirer;

FIG. 12 is a diagram illustrating a modeled vessel;

FIG. 13 is a diagram illustrating a relation between an example of a waveform of a pulse wave and a radius of a modeled cylindrical tube;

FIG. 14 is a diagram schematically illustrating changes of a radius of a cylindrical tube with changes of a volume of a vessel from a point Pₛ to a point P_{L};

FIG. 15 is a diagram schematically illustrating a flow rate Q_{S} from the point Pₛ to a point P_{H} through a point P_{E};

FIG. 16 is a diagram schematically illustrating a flow rate Q_{HD} from the point P_{H} to a point P_{D};

FIG. 17 is a diagram illustrating an example of information acquired by a characteristic point processor 52 from a pulse wave yᵢ;

FIG. 18 is a diagram illustrating measurement data of a subject whose blood pressure according to a second blood pressure is relatively high;

FIG. 19 is a diagram illustrating measurement data of a subject whose blood pressure according to the second blood pressure is relatively low;

FIG. 20 is a block diagram illustrating a configuration example of a non-pressurizing blood pressure gauge;

FIG. 21 is a diagram illustrating an example of a wristwatch-type blood pressure processing apparatus; and

FIG. 22 is a flowchart illustrating an example of control on calibration processing.

### DETAILED DESCRIPTION

An embodiment of the present invention has been achieved in view of these circumstances and has an object to provide an electronic apparatus, and a method that can easily and accurately acquire the blood pressure of a subject.

According to the present embodiment, an electronic apparatus comprising a processor configured to generate a calibration period corresponding to a pressurization period of a pressurizer for a subject using a received light signal based on scattered light scattered in a body of the subject when an optical signal of a predetermined frequency band is applied thereto, acquire a first blood pressure based on the received light signal in the calibration period, and to generate calibration information using a reference blood pressure measured on a basis of pressurization of the pressurizer, and the first blood pressure.

The embodiment of the present invention is explained below with reference to the drawings. While characteristic configurations and operations of the electronic apparatus (a blood pressure processing apparatus) are mainly explained in the following embodiment, configurations and operations omitted in the following explanations can be included in the blood pressure processing apparatus.

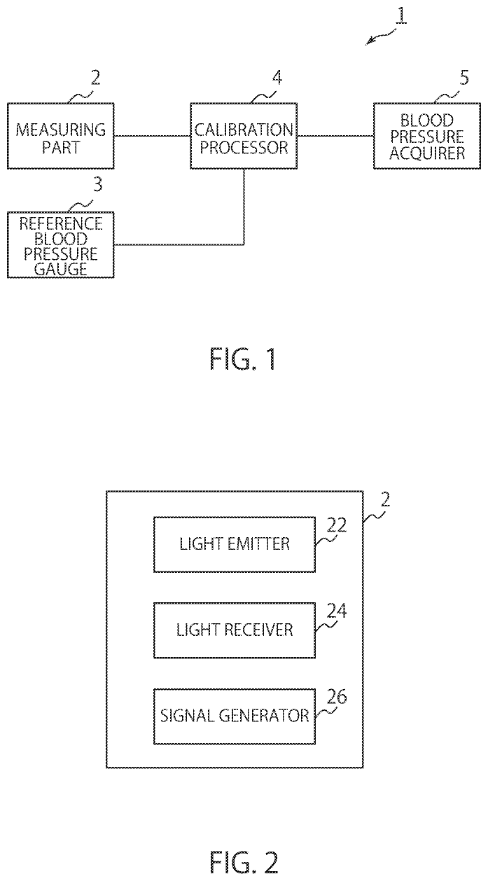

FIG. 1 is a block diagram illustrating a schematic configuration of a blood pressure processing apparatus 1 according to the present embodiment. The blood pressure processing apparatus 1 includes a measuring part 2, a reference blood pressure gauge 3, a calibration processor 4, and a blood pressure acquirer 5.

The measuring part 2 generates a measurement signal based on a scattered signal that is scattered within the body of a subject when an optical signal of a predetermined frequency band is applied thereto. Details of the measuring part 2 will be described later.

The reference blood pressure gauge 3 is, for example, a cuff sphygmomanometer that measures the blood pressure of a subject in a state where an arm of the subject is pressurized by a pressurizer that pressurizes the arm of the subject. The pressurizer is, for example, a cuff and is placed on a side of the subject nearer the heart than the measurement place of the measuring part 2. The reference blood pressure gauge 3 supplies a measurement signal including information on the measured blood pressure to the calibration processor 4. Furthermore, the reference blood pressure gauge 3 is configured to be removable from the pressure blood processing apparatus 1. While being, for example, a cuff sphygmomanometer, the reference blood pressure gauge 3 according to the present embodiment is not limited thereto. For example, a sphygmomanometer that has a pressurizer on a side of a subject nearer the heart than the measuring place of the measuring part 2 suffices.

The calibration processor 4 generates calibration information using a reference blood pressure measured during a cuff pressurization period, and a first blood pressure measured using the measurement signal during a calibration period. The blood pressure acquirer 5 acquires the first blood pressure of a subject on the basis of a pulse wave to be generated by the measuring part 2. Details of the calibration processor 4 and the blood pressure acquirer 5 are also described later.

FIG. 2 is a block diagram illustrating a configuration example of the measuring part 2. The measuring part 2 includes a light emitter 22, a light receiver 24, and a signal generator 26. The light emitter 22 has, for example, an LED (Light Emitting Device) that emits an optical signal of a certain wavelength band (green, a near-infrared band, or the like). The light receiver 24 receives a signal that is the optical signal from the light emitter 22 having been absorbed or reflected and scattered in the body of a subject.

The signal generator 26 generates the measurement signal on the basis of the received light signal. The signal generator 26 also can generate a pulse wave signal with respect to one heartbeat. When the amount of emitted light of the optical signal varies, the amount of received light of the received light signal also varies. Therefore, the signal generator 26 separates the received light signal into a DC component and an AC component and generates a pulse wave signal on the basis of the AC/DC ratio. Accordingly, the generated pulse wave signal is non-dimensional data.

The signal generator 26 has an amplifier that amplifies a signal, and an AD converter that converts an analog signal into a digital signal, and converts the measurement signal and the pulse wave signal into digital signals. The blood volume pulse is hereinafter referred to also simply as “pulse”.

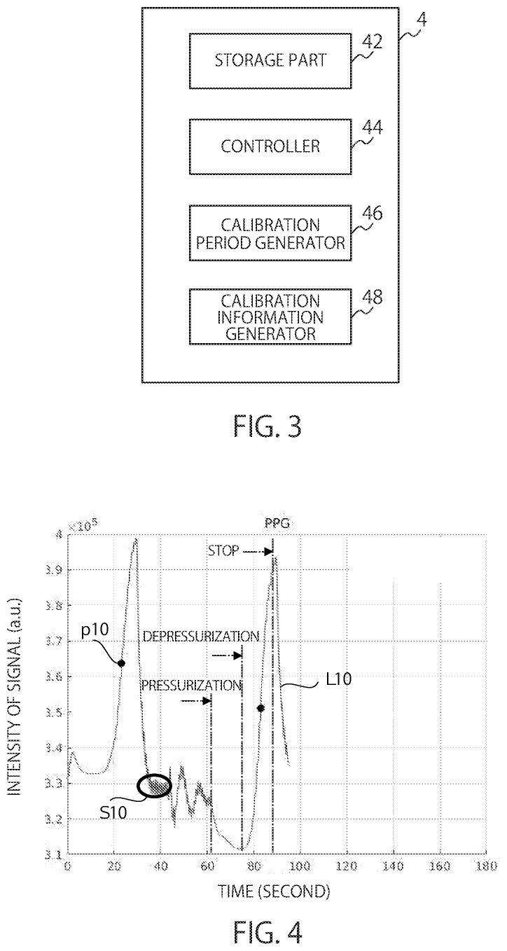

FIG. 3 is a block diagram illustrating a configuration example of the calibration processor 4. The calibration processor 4 includes a storage part 42, a controller 44, a calibration period generator 46, and a calibration information generator 48.

The storage part 42 stores therein the measurement signal generated in time series, the first blood pressure to be generated by the blood pressure acquirer 5, and the blood pressure to be generated by the reference blood pressure gauge 3, which are associated in time series. The controller 44 has an internal clock and controls the entire blood pressure processing apparatus 1.

The calibration period generator 46 generates a calibration period based on a pressurization period where the pressurizer of the reference blood pressure gauge 3 pressurizes a subject. Characteristics of the measurement signal, including the pressurization period to be used by the calibration period generator 46 are explained below with reference to FIGS. 4 to 8. A measurement signal L10 is generated by the measuring part 2 as described above.

FIG. 4 is a diagram illustrating an example of the measurement signal L10 of a subject A. The horizontal axis represents the time and the vertical axis represents the magnitude of the measurement signal L10. Points p10 in FIG. 4 are time points where a time difference value, for example, a first-order differential value in the pressurization period of the pressurizer (the cuff) has the maximum value. A signal S10 indicates an example of a region where the pulse wave based on pulsation is observed in a pulse manner.

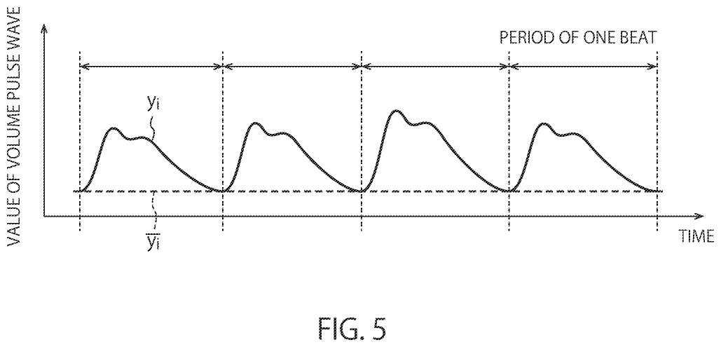

FIG. 5 is a diagram illustrating an example of the volume pulse wave based on the signal S10. The vertical axis represents the value of the volume pulse wave and the horizontal axis represents the time. As illustrated in FIG. 5, the pulse wave signal is generated by the signal generator 26 and the pulse wave repeats fluctuation with respect to each heartbeat. A pulse wave yᵢ of an ith beat is constituted of

y̅ᵢ

which is an AC component and a DC component periodically fluctuating. In this way, a pulsed wave in regions such as the region of the signal S10 pulsates with respect to each heartbeat and has information on the pulse wave.

As illustrated again in FIG. 4, in the measurement signal L10 of the subject A, the intensity of the measurement signal L10 decreases when the pressurization of the cuff is started. When depressurization of the cuff is started, the intensity of the measurement signal L10 increases. The pulse wave in a pulsed manner is suppressed in the pressurization period of the cuff. Therefore, the measurement of the blood pressure by the blood pressure acquirer 5 using the information on the pulse wave in the pulsed manner illustrated in FIG. 4 is difficult in the pressurization period of the cuff. Meanwhile, the cuff sphygmomanometer is standard in the medical industry and the value of the blood pressure using the volume pulse wave is required to be coincident with the value of the reference blood pressure gauge 3. Accordingly, there is a need to obtain a region for blood pressure measurement by the blood pressure acquirer 5 having a high correlation with the reference blood pressure to be acquired during the cuff pressurization period.

The pressurization period of the reference blood pressure gauge 3 varies among individuals and the pressurization period is, for example, around 40 seconds. The reference blood pressure gauge 3 measures a set of the maximum blood pressure and the minimum blood pressure, and the average blood pressure in this period. Meanwhile, the blood pressure acquirer 5 can measure at least the maximum blood pressure and the minimum blood pressure with respect to each beat of a subject.

In FIG. 4, the cuff pressure is applied twice or three times, every approximately 60 seconds. The blood is thus pumped from the heart with increases of the pressure applied by the cuff, whereby the amplitude of the measurement signal L10 decreases. Therefore, the baseline (an average amount of absorbed light) is likely to decline. On the other hand, when the pressure turns to decrease, the baseline rises. It was found by the applicant that the point p10 in FIG. 4 always arises in the depressurization period between a time point when the depressurization of the cuff is started and a time point when the pressurization is stopped. As described above, it was found that the baseline generally does not exhibit such a steep increase unless the depressurization of the cuff is started.

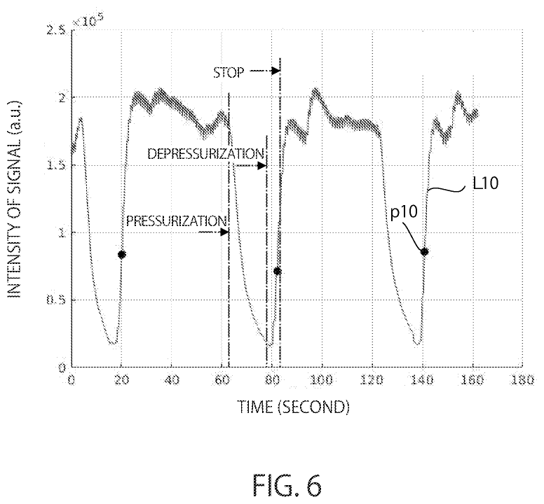

FIG. 6 is a diagram illustrating an example of the measurement signal L10 of a subject B. Similarly to FIG. 4, the horizontal axis represents the time and the vertical axis represents the magnitude of the measurement signal L10. As illustrated in FIG. 6, in the measurement signal L10 of the subject B, the intensity of the measurement signal L10 has a tendency of decreasing more than that of the subject A when the pressurization of the cuff is started. On the other hand, the intensity of the measurement signal L10 steeply increases similarly to the subject A when the depressurization of the cuff is started. Therefore, the point p10 in FIG. 6 always arises in the depressurization period also in FIG. 6.

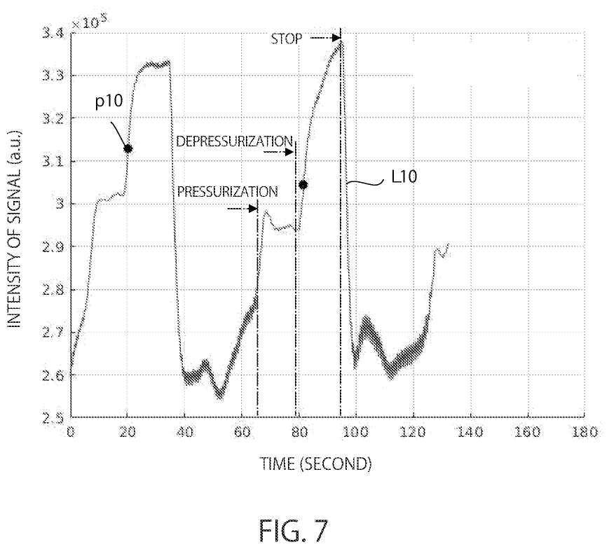

FIG. 7 is a diagram illustrating an example of the measurement signal L10 of a subject C. Similarly to FIG. 4, the horizontal axis represents the time and the vertical axis represents the magnitude of the measurement signal L10. As illustrated in FIG. 7, in the measurement signal L10 of the subject C, the intensity of the measurement signal L10 has a tendency of temporarily increasing when the pressurization of the cuff is started. On the other hand, when the depressurization of the cuff is started, the intensity of the measurement signal L10 steeply increases similarly to the subjects A and B. Therefore, the point p10 in FIG. 7 always arises in the depressurization period between a time point when the depressurization of the cuff is started and a time point when the pressurization is stopped also in FIG. 7.

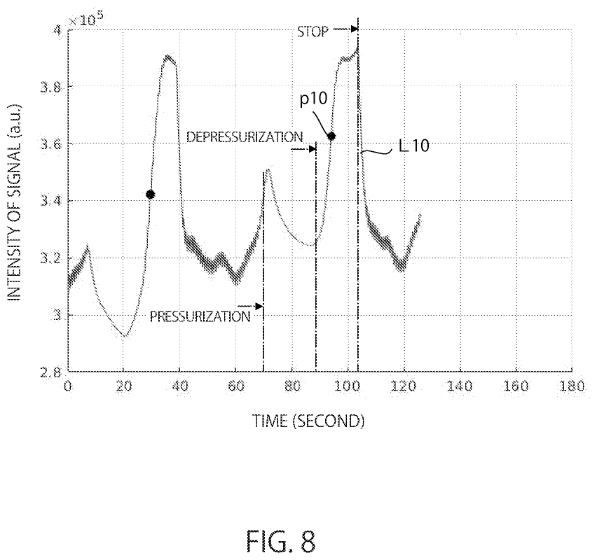

FIG. 8 is a diagram illustrating an example of the measurement signal L10 of a subject D. Similarly to FIG. 4, the horizontal axis represents the time and the vertical axis represents the magnitude of the measurement signal L10. As illustrated in FIG. 8, in the measurement signal L10 of the subject D, the intensity of the measurement signal L10 has a tendency of temporarily increasing and then decreasing when the pressurization of the cuff is started. On the other hand, when the depressurization of the cuff is started, the intensity of the measurement signal L10 steeply increases similarly to the subjects A, B, and C. Therefore, the point p10 in FIG. 8 always arises in the depressurization period also in FIG. 8.

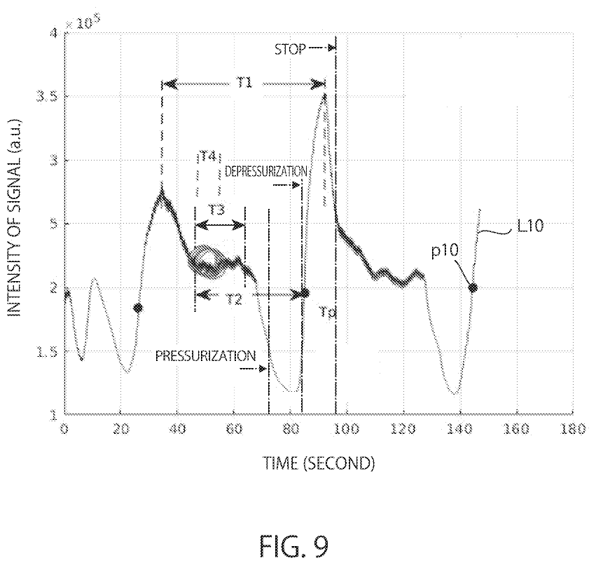

FIG. 9 is a diagram illustrating an example of the measurement signal L10 during a calibration period. The horizontal axis represents the time and the vertical axis represents the magnitude of the measurement signal L10. As illustrated in FIG. 9, for example, the blood pressure processing apparatus 1 performs the measurement of the reference blood pressure by the reference blood pressure gauge 3 at measurement intervals T1=60 seconds, and stores the measurement signal L10, the first blood pressure to be generated by the blood pressure acquirer 5, and the blood pressure to be generated by the reference blood pressure gauge 3 associated in time series in the storage part 42 during the calibration period.

The calibration period generator 46 generates the calibration period based on the pressurization period of the cuff (the pressurizer) using the characteristics that the point p10 in FIGS. 4 to 8 always arises between a time point when the depressurization of the cuff is started and a time point when the pressurization is stopped as explained with reference to FIGS. 4 to 8. More specifically, the calibration period generator 46 performs smoothing processing of the measurement signal L10 stored in the storage part 42. Accordingly, the measurement signal L10 where the pulsation and noise are suppressed is generated from the measurement signal L10. Next, the calibration period generator 46 obtains a time point p10 where a value based on the time difference value of the measurement signal L10, for example, the first-order differential value has the maximum value. Subsequently, the calibration period generator 46 sets a predetermined period T4 from a time a predetermined period T2 before the time point p10 until a time a predetermined time T3 thereafter as the calibration period. It is assumed, for example, that T2=37 seconds, T3=15 seconds, and T4=5 seconds. That is, five seconds within a period from a time 37 seconds before the time point p10 until a time 22 seconds before the time point p10 are set as the calibration period. Circles in FIG. 9 indicate examples of five seconds in the period from the time 37 seconds before the time point p10 until the time 22 seconds before the time point p10.

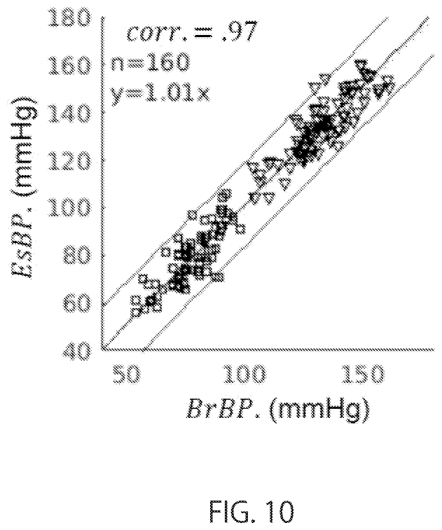

FIG. 10 is a diagram illustrating a relation between the first blood pressure to be generated by the blood pressure acquirer 5 in the calibration period and the reference blood pressure to be generated by the reference blood pressure gauge 3. The horizontal axis represents the maximum blood pressure of the reference blood pressure and the vertical axis represents the maximum blood pressure of the first blood pressure. For example, a relation between the first blood pressure to be generated by the blood pressure acquirer 5 during five seconds within the period from the time 37 seconds before the time point p10 until the time 22 seconds before, and the reference blood pressure to be generated by the reference blood pressure gauge 3 is illustrated. In this way, it is experimentally verified that the correlation value during the calibration period is 0.97 and that the first blood pressure and the reference blood pressure have a high correlation.

The calibration information generator 48 generates the calibration information using the reference blood pressure measured by the reference blood pressure gauge 3 on the basis of the pressurization of the pressurizer of the reference blood pressure gauge 3 and the first blood pressure of the blood pressure acquirer 5. For example, the calibration information generator 48 computes the average value of ratios between the maximum blood pressure of the first blood pressure generated during the calibration period and the maximum blood pressure of the corresponding reference blood pressure, and the difference value between the maximum blood pressure of the first blood pressure and the maximum blood pressure of the corresponding reference blood pressure. Similarly, the calibration information generator 48 computes the average value of ratios between the minimum blood pressure of the first blood pressure generated during the calibration period and the minimum blood pressure of the corresponding reference blood pressure, and the difference value between the minimum blood pressure of the first blood pressure and the minimum blood pressure of the corresponding reference blood pressure.

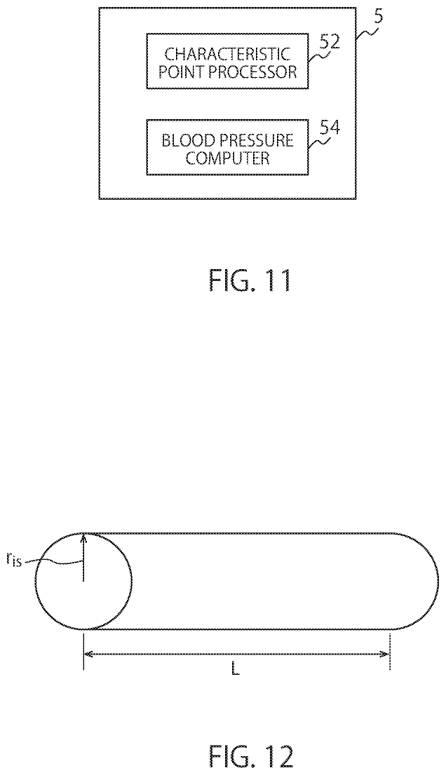

FIG. 11 is a block diagram illustrating a configuration example of the blood pressure acquirer 5. As illustrated in FIG. 11, the blood pressure acquirer 5 can be calibrated with the calibration information to be generated by the calibration information generator 48 and acquires the blood pressure of a subject on the basis of the pulse wave signal. The blood pressure acquirer 5 includes a characteristic point processor 52 and a blood pressure computer 54.

A model example to be used in the blood pressure acquirer 5 is explained first with reference to FIGS. 12 to 16. FIG. 12 is a diagram illustrating a modeled vessel. The model of a vessel illustrated in FIG. 12 is approximated by a cylindrical tube having a radius rᵢₛ and a length L. Blood pressure fluctuation is fluctuation of pressure applied to the vessel wall by the blood pumped from the heart. This blood pressure fluctuation is linked with the pulse wave yᵢ.

A relation among the pressure difference ΔP, the flow rate Q, and the resistance R of the cylindrical tube is derived from Darcy's law and is represented by expression (1).

ΔP=QR (1)

The blood pressure acquirer 5 computes a value corresponding to the flow rate Q and the resistance R using the pulse wave yᵢ, for example, on the basis of the cylindrical tube model to acquire the blood pressure of a subject. While being based on the cylindrical tube model, the blood pressure acquirer 5 according to the present embodiment is not limited thereto. For example, a non-pressurizing blood pressure gauge based on other algorisms may be used.

The blood pressure in humans is generally evaluated using the systolic blood pressure (the maximum blood pressure) SBP that is the maximum pressure in the vessel during systole of the heart, the diastolic blood pressure (the minimum blood pressure) DBP that is the minimum pressure in the vessel during diastole of the heart, and the pulse pressure PP obtained by subtracting the systolic blood pressure from the diastolic blood pressure.

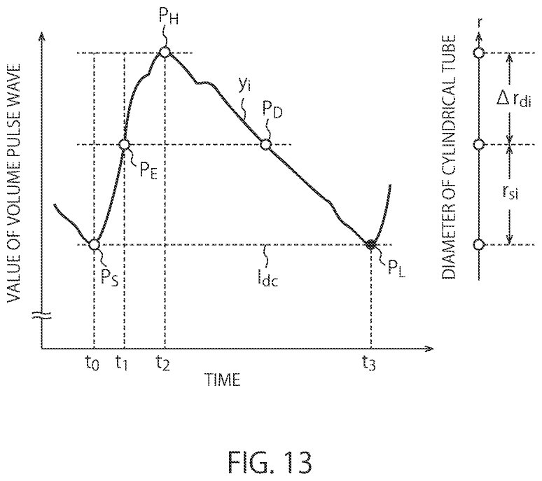

FIG. 13 is a diagram illustrating a relation between an example of the waveform of a pulse wave and the radius of a modeled cylindrical tube. The left drawing is a diagram illustrating an example of the waveform of a normal pulse wave of one beat. The vertical axis represents the value of the pulse wave and the horizontal axis represents the time. The right drawing is a diagram illustrating the radius of a modeled cylindrical tube. Volume changes of the vessel are represented by the radius rₛᵢ and Δr_{di} being changes. A point P_{D} is a point indicating a value of the volume pulse wave that is the same as that of a point P_{E} between a point P_{H} and a point P_{L}. I_{dc} is a DC component of the volume pulse wave.

The amplitude of a normal pulse wave yᵢ starts at a position (t₀) of the bottom, the amplitude substantially monotonously increases to reach a maximum peak (t₂), and the amplitude then monotonously decreases to reach a position (t₃) of the bottom and ends. The index i is a number for identifying each pulse in volume pulse wave data. That is, this indicates data corresponding to an ith pulse wave. While computing for each beat is performed in the computing according to the present embodiment, the computing is not limited thereto and the computing may be performed, for example, by averaging data of several beats.

t₁ is a time when a value obtained by first-order differential of the pulse wave yᵢ with the time becomes largest between t₀ and t₂. The time t₁ corresponds to a displacement equilibrium point of an equation of viscoelastic motion.

The points P_{E}, P_{H}, and P_{L} are points corresponding to the times t₁, t₂, and t₃, respectively. The time t₁ according to the present embodiment corresponds to a first time, the time t₂ corresponds to a second time, the time t₀ corresponds to a third time, and the time t₃ corresponds to a fourth time. The index i is a number for identifying each pulse in the volume pulse data. That is, this indicates data corresponding to an ith pulse wave. The time of the point P_{D} corresponds to a fifth reference time.

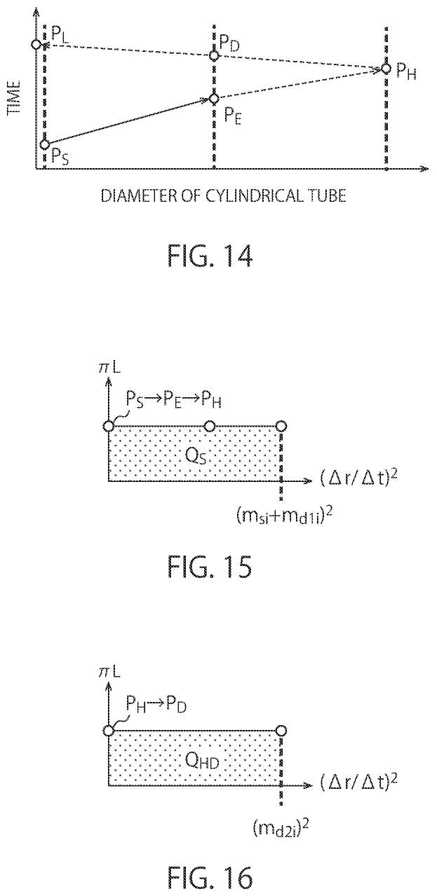

FIG. 14 is a diagram schematically illustrating changes of the radius of a cylindrical tube with changes of the volume of the vessel from a point P_{S} to the point P_{L}. That is, FIG. 14 illustrates changes of the radius of the cylindrical tube associated with the pulse wave of one beat. The vertical axis represents the time and the horizontal axis represents the changes of the radius from the point P_{S}. The radius increases from the point P_{S} to the point P_{H} and thereafter decreases with passage of time.

FIG. 15 is a diagram schematically illustrating a flow rate Q_{S} from the point P_{S} to the point P_{H} through the point P_{E}. FIG. 16 is a diagram schematically illustrating a flow rate Q_{HD} from the point P_{H} to the point P_{D}. The horizontal axis represents the square of the average change rate (Δr/Δt) of the vessel radius r and the vertical axis represents the multiplication value of the length L and n. In FIG. 15, mₛᵢ is the average change rate of the radius from the point P_{S} to the point P_{E} and m_{d1i} is the average change rate of the radius from the point P_{E} to the point P_{H}. In FIG. 16, m_{d2i} is the average change rate of the radius of the point P_{D} and the radius of the point P_{H}. The flow rate Q_{HD} according to the present embodiment corresponds to a first value, the resistance R corresponds to a second value, and the flow rate Q_{S} corresponds to a third value.

In the present embodiment, the systolic blood pressure SBP is computed using the flow rate Q_{S}. Dilation of the vessel radius until the point P_{E}, and the reconstruction to the point P_{L} are known as the Windkessel effect.

From the point P_{E}, the cardiac output force becomes progressively lower, and then restoring force and damping force gradually become dominant. That is, in the present embodiment, the modeling is performed by the flow considering the cardiac output force between the point P_{E} and P_{H}. It is considered that a stronger cardiac output force is exerted also in the range from the point P_{E} to the point P_{H} on some subjects. When the measurement is performed to people including these subjects, use of the flow rate Q_{S} can further improve the measurement accuracy of the systolic blood pressure SBP. It is experimentally verified that the measurement accuracy of the systolic blood pressure SBP of ordinary people is not reduced even when the flow rate Q_{S} is used.

Meanwhile, it is considered that a point where the cardiac output force is weakened is shifted toward the point P_{L} in the case of the person who has a stronger cardiac output force in the range from the point P_{E} to the point P_{H}. Since the diastolic blood pressure is a lower limit of force produced by the Windkessel effect, the diastolic blood pressure DBP is modeled by shifting the point where the cardiac output force is weakened to the point P_{H} and using the flow rate Q_{D} in the range from the point P_{H} to the point P_{D}. Particularly, the flow rate Q_{D} is computed on the basis of the flow rate Q_{HD}. It is experimentally verified that also the measurement accuracy of the diastolic blood pressure DBP of ordinary people is not lowered even when the flow rate Q_{HD} is used.

The foregoing is explanations of the model to be used by the blood pressure acquirer 5 according to the present embodiment. A detailed processing example of the blood pressure acquirer 5 is explained below.

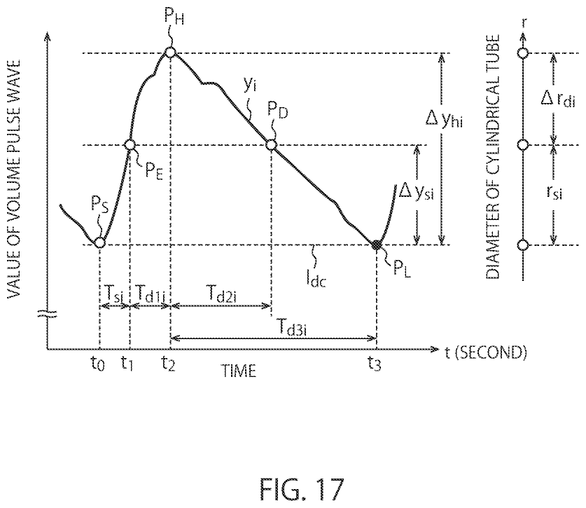

FIG. 17 is a diagram illustrating an example of information acquired by the characteristic point processor 52 from the pulse wave yᵢ. The vertical axis represents the value of the pulse wave and the horizontal axis represents the time. The right drawing is a diagram illustrating the radius of a modeled cylindrical tube. Volume changes of the vessel are represented by the radius rₛᵢ and Δr_{di} being changes.

The characteristic point processor 52 computes a first difference value Δyₛᵢ by subtracting the DC component I_{dc} from a value of the pulse wave yᵢ at the time t₁, and a second difference value Δyₕᵢ by subtracting the DC component I_{dc} from a value of the pulse wave yᵢ at the time t₂.

The characteristic point processor 52 also computes a time T_{d2i} using expression (2). The time T_{d2i} is a time between the point P_{D} and the point P_{H}. A time Tₛᵢ is a time obtained by subtracting the time to from the time t₁, a time T_{d1i} is a time obtained by subtracting the time t₁ from the time t₂, and a time T_{d3i} is a time obtained by subtracting the time t₂ from the time t₃. That is, the characteristic point processor 52 acquires a time of the point P_{D} indicating a value equivalent to the pulse wave yᵢ at the time t₁ in a period from the time t₂ to the time t₃ of the pulse wave yᵢ as the fifth reference time and computes a time between the time t₂ and the fifth reference time as the time T_{d2i}.

$${T}_{d 2 i} \sim \frac{( {T}_{si} + {T}_{d 1 i} ) {{T}_{d 3 i} ( Δ {y}_{hi} - Δ {y}_{si} )}^{2}}{{T}_{d 1 i} Δ {y}_{hi}^{2}} . \qquad ( 2 )$$

With respect to the volume corresponding to the point P_{L}, Δyₛᵢ/I_{dc} is proportional to the volume at the points P_{E} and P_{D}, and Δyₕᵢ/I_{c} is similarly proportional to the volume at the point P_{H}. G is a proportional constant and I_{dc} is a value of the DC component of the pulse wave.

When the radius of the cylindrical tube changes from rₛᵢ to rₛᵢ+Δr_{di}, Δr_{di} can be computed by expressions (3) to (5) using the radius rₛᵢ at the point P_{E}.

$$\begin{matrix}{V}_{i} = G Δ {y}_{si} / {I}_{dc} & ( 3 ) \\ Δ {V}_{i} = G \frac{Δ {y}_{hi}}{{I}_{dc}} - {V}_{i} & ( 4 ) \\ Δ {r}_{di} = \frac{{r}_{si}}{2} \frac{Δ {V}_{i}}{{V}_{i}} & ( 5 )\end{matrix}$$

The blood pressure computer 54 computes the radius rₛᵢ and Δr_{di} using the expressions (6) and (7). L is the length of the modeled cylindrical tube.

$$\begin{matrix}{r}_{si} = \sqrt{\frac{G}{L}} \sqrt{\frac{Δ {y}_{si}}{π {I}_{dc}}} & ( 6 ) \\ Δ {r}_{di} = \frac{1}{2} \frac{Δ {y}_{hi} - Δ {y}_{si}}{Δ {y}_{si}} \sqrt{\frac{G}{L}} \sqrt{\frac{Δ {y}_{si}}{π {I}_{dc}}} & ( 7 )\end{matrix}$$

The blood pressure computer 54 computes the average change rate mₛᵢ using expression (8).

$${m}_{si} = \frac{{r}_{si}}{{T}_{si}} \qquad ( 8 )$$

The blood pressure computer 54 computes the average change rates m_{d1i} and m_{d2i} using expression (9) and (10), respectively.

$$\begin{matrix}{m}_{d 1 i} = \frac{Δ {r}_{di}}{{T}_{d 1 i}} & ( 9 ) \\ {m}_{d 2 i} = \frac{Δ {r}_{di}}{{T}_{d 2 i}} & ( 10 )\end{matrix}$$

The blood pressure computer 54 computes the flow rate Q_{Si} on the basis of the average change rates m_{d1i} and m_{d2i} using expression (11).

Qₛᵢ=πL(mₛᵢ+m_{di})² (11)

The blood pressure computer 54 computes a resistance Ri using expression (12). In this expression, Vᵢ is the cubic volume of the modeled cylindrical tube, and Vᵢ(t₁) is the cubic volume of the modeled cylindrical tube at the time t₁. That is, I_{dcs} corresponds to

y̅ᵢ

in the present embodiment.

$${R}_{i} = \frac{{I}_{dc} - {V}_{i}}{\frac{{dV}_{i} ( {t}_{1} )}{dt}} \qquad ( 12 )$$

The blood pressure computer 54 computes the flow rate Q_{Di} from the point P_{H} to the point P_{L} on the basis of expression (13) using the resistance Rᵢ and compliance C.

$$\sqrt{{Q}_{Di}} = {Q}_{HDi} {e}^{- {T}_{d 3 i} / {R}_{di} C} = π {L ( {m}_{d 2 i} )}^{2} {e}^{- {T}_{d 3 i} / {R}_{di} C} = G \frac{{( Δ {y}_{hi} - Δ {y}_{si} )}^{2}}{4 {I}_{dc} Δ {{y}_{si} ( {T}_{d 2 i} )}^{2}} {e}^{- {T}_{d 3 i} / {R}_{di} C} \qquad ( 13 )$$

The resistance R_{di} is obtained from expressions (14) and (15).

$$\begin{matrix}{y}_{di}^{′} = \frac{{y}_{i} ( {t}_{2} + {T}_{d 2 i} + 1 / fs ) - {y}_{i} ( {t}_{2} + {T}_{d 2 i} )}{1 / fs} & ( 14 ) \\ {R}_{di} = \frac{\vert {y}_{di}^{′} \vert}{{y}_{i}^{′}} & ( 15 )\end{matrix}$$

The blood pressure computer 54 computes the diastolic blood pressure (the minimum blood pressure) DBP and the systolic blood pressure (the maximum blood pressure) SBP every i beats on the basis of expressions (16) and (17).

ln DBPᵢ=a₁ ln Q_{Di}+a₂ ln R_{di}+α (16)

ln SBPᵢ=b₁ ln Qₛᵢ+b₂ ln R_{di}+β+ln DBPᵢ (17)

where a₁, a₂, b₁, b₂, α, and β are constants.

K1 and K2 are calibration factors which are examples of the calibration information generated by the calibration processor 4. An initial state in which K1=1 and K2=1 according to the present embodiment corresponds to the first blood pressure.

DBPᵢ=K1 exp(a₁ ln Q_{Di}+a₂ ln R_{di}+α) (18)

SBPᵢ=K2 exp(b₁ ln Qₛᵢ+b₂ ln R_{di}+β+ln DBPᵢ) (19)

The diastolic blood pressure (the minimum blood pressure) DBP and the systolic blood pressure (the maximum blood pressure) SBP after substitution of K1 and K2 generated by the calibration processor 4 correspond to the second blood pressure.

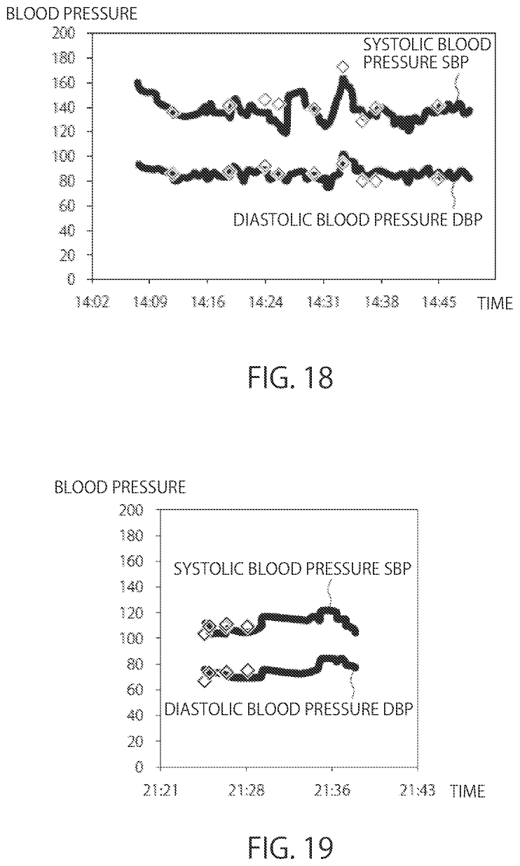

FIG. 18 is a diagram illustrating measurement data of a subject whose blood pressure according to the second blood pressure is relatively high. FIG. 19 is a diagram illustrating measurement data of a subject whose blood pressure according to the second blood pressure is relatively low. The vertical axis represents the blood pressure and the horizontal axis represents the time. Rhomboid marks indicate measurement values of the reference blood pressure gauge 3 and solid lines indicate data of the second blood pressure. In both cases, values measured by the blood pressure processing apparatus 1 according to the present embodiment are satisfactorily coincident with data of the reference blood pressure gauge 3 measured as comparative targets.

In this way, the blood pressure computer 54 acquires the diastolic blood pressure DBP on the basis of the flow rate Q_{HD} (the first value) corresponding to the blood flow rate of a subject in the period T_{d2i} (the first period) within a period from the first time t₁ when the value obtained by first-order differential of the pulse wave yᵢ with the time becomes largest until the fourth time t₃ when the next pulse wave rises, and R (the second value) corresponding to the blood resistance of the subject. The blood pressure computer 54 acquires the systolic blood pressure SBP further on the basis of the flow rate Qₛ (the third value) corresponding to the blood flow rate of the subject in a period (Tₛᵢ+T_{d1i}) (the second period) within a period from the third time t₀ when the pulse wave rises until the second time t₂ of the maximum peak of the pulse wave. The first time according to the present embodiment corresponds to the first reference time, the second time corresponds to the fourth reference time, the third time corresponds to the third reference time, and the fourth time corresponds to the second reference time. Since calibrated diastolic blood pressure DBPi and systolic blood pressure SBPi are obtained on the basis of the expressions (18) and (19) in the present embodiment, the blood pressure can be detected easily and accurately.

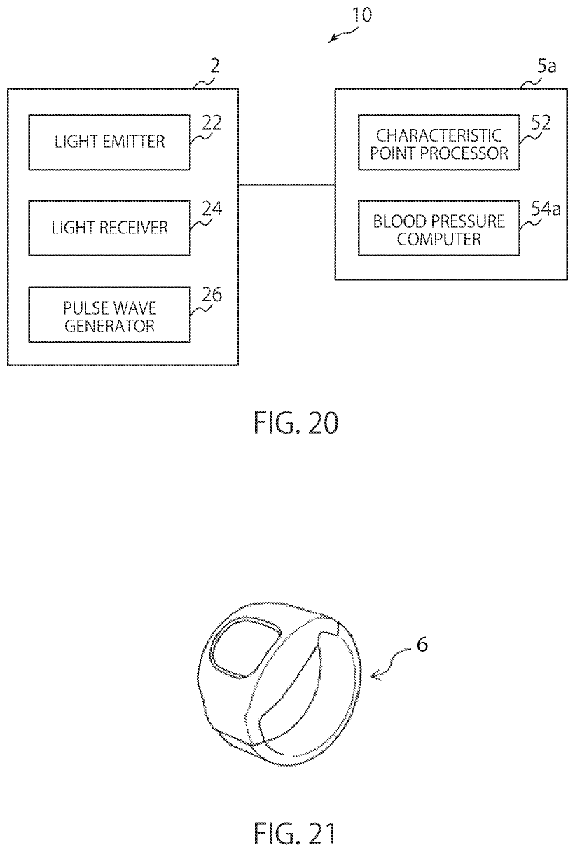

FIG. 20 is a block diagram illustrating a configuration example of a non-pressurizing blood pressure measuring apparatus 10 including the measuring part 2 and a calibrated second blood pressure acquirer 5a. A blood pressure computer 54a of the second blood pressure acquirer 5a is different from the blood pressure computer 54 (see FIG. 11) of the blood pressure acquirer 5 in using calibrated K1 and K2 generated by the calibration processor 4 in the expressions (18) and (19). The measuring part 2 of the blood pressure measuring apparatus 10 may be configured to generate the pulse wave without generating the measurement signal. The blood pressure measuring apparatus 10 is also called sphygmomanometer.

As illustrated in FIG. 20, the non-pressurizing blood pressure measuring apparatus 10 may be constituted using the calibrated second blood pressure acquirer 5a. The blood pressure measuring apparatus 10 can be incorporated into, for example, a wristwatch-type biometric measurement device 6 as illustrated in FIG. 21. The biometric measurement device 6 may be placed at an upper arm part or a chest part.

Alternatively, the blood pressure processing apparatus 1 (see FIG. 1) in a state where the reference blood pressure gauge is removed therefrom may be incorporated into, for example, the wristwatch-type biometric measurement device 6 as illustrated in FIG. 21. In this case, the blood pressure acquirer 5 after substitution of K1 and K2 generated by the calibration processor 4 is used. When the blood pressure processing apparatus 1 (see FIG. 1) is incorporated into the wristwatch-type biometric measurement device 6, recalibration can be performed by connecting the reference blood pressure gauge 3 thereto.

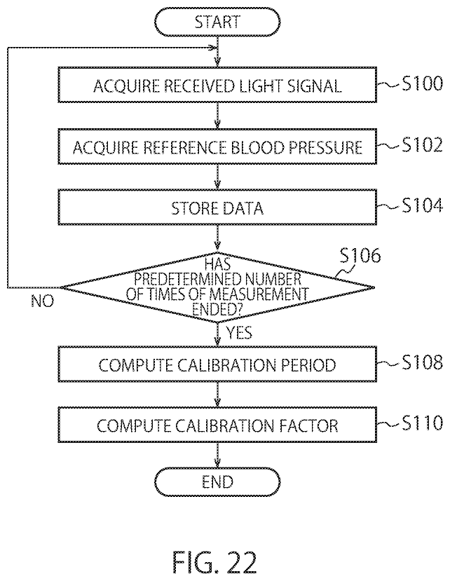

FIG. 22 is a flowchart illustrating an example of control on the calibration processing executed by the controller 44 of the blood pressure processing apparatus 1. As illustrated in FIG. 22, under the control of the controller 44, the measuring part 2 first generates the measurement signal and the pulse wave signal based on the received light signal scatted in the body of a subject when an optical signal of a predetermined frequency band is applied (Step S100).

Next, in synchronization with the measurement of the measuring part 2, the reference blood pressure gauge 3 generates a reference blood pressure of the subject, for example, in a cycle of every 60 seconds (Step S102). Subsequently, the storage part 42 stores therein the data associated with each other in time series (Step S104).

Next, the controller 44 determines whether a predetermined number of times of the measurement by the reference blood pressure gauge 3 has ended (Step S106). The controller 44 repeats the processing from Step S100 when determining that the predetermined number of times of the measurement has not ended (NO at Step S106).

On the other hand, when the controller 44 determines that the predetermined number of times of the measurement has ended (YES at Step S106), the controller 44 causes the calibration period generator 46 to generate the calibration period with respect to each measurement cycle, using the data stored in the storage part 42 (Step S108). Subsequently, the controller 44 causes the blood pressure acquirer 5 to compute the diastolic blood pressure (the minimum blood pressure) DBP and the systolic blood pressure (the maximum blood pressure) SBP in the calibration period with respect to each measurement cycle, and causes the calibration information generator 48 to generate the ratios thereof to the reference blood pressure as the calibration factors K1 and K2, respectively, for each measurement. The controller 44 causes the calibration information generator 48 to generate the average values of the calibration factors K1 and K2 as definitive calibration factors K1 and K2 (Step S110) and ends the whole processing.

As described above, according to the present embodiment, the calibration period generator 46 generates the calibration period based on the pressurization period of the pressurizer for a subject on the basis of the received light signal, and the calibration information generator 48 generates the calibration information using the first blood pressure generated by the blood pressure acquirer 5 in the calibration period and the reference blood pressure. Since the first blood pressure generated in the calibration period based on the pressurization period has a high correlation with the reference blood pressure, the calibration information can be generated more accurately. Accordingly, the blood pressure measuring apparatus 10 calibrated with the calibration information can generate the measurement value more coincident with the reference blood pressure.

At least a part of the blood pressure processing apparatus 1 can be constituted by hardware or software. When it is constituted by software, the blood pressure processing apparatus 1 can be configured such that a program for realizing at least a part of the functions of the blood pressure processing apparatus 1 is stored in a recording medium such as a flexible disk or a CD-ROM, and the program is read and executed by a computer. The recording medium is not limited to a detachable device such as a magnetic disk or an optical disk, and can be a fixed recording medium such as a hard disk device or a memory.

Further, at least a part of the blood pressure processing apparatus 1 can be implemented by one or more processors. The processor is, for example, one or more electronic circuits including a control device and an arithmetic device. The electronic circuit is realized by an analog or digital circuit or the like. For example, a general-purpose processor, a central processing unit (CPU), a microprocessor, a digital signal processor (DSP), an ASIC, an FPGA, and a combination thereof are possible. At least a part of the blood pressure processing apparatus 1 is, for example, a part of the signal generator 26, the calibration processor 4 (the controller 44, the calibration period generator 46, the calibration information generator 48), and the blood pressure acquirer 5 (the characteristic point processor 52, blood pressure computer 54). Further, one component can be implemented separately in a plurality of processors.

While certain embodiments have been described, these embodiments have been presented by way of example only, and are not intended to limit the scope of the inventions. Indeed, the novel embodiments described herein may be embodied in a variety of other forms and various omissions, substitutions, and changes may be made without departing from the spirit of the inventions. The embodiments and their modifications are intended to be included in the scope and the spirit of the invention and also in the scope of the invention and their equivalents described in the claims.

1. An electronic apparatus comprising a processor configured to:
generate a calibration period corresponding to a pressurization period of a pressurizer for a subject using a received light signal based on scattered light scattered in a body of the subject when an optical signal of a predetermined frequency band is applied thereto;
acquire a first blood pressure based on the received light signal in the calibration period; and
to generate calibration information using a reference blood pressure measured on a basis of pressurization of the pressurizer, and the first blood pressure.

2. The electronic apparatus according to claim 1, wherein the processor is configured to generate the calibration period on a basis of characteristics of a measurement signal based on the received light signal, the characteristics indicating a time point in a depressurization period from a time point when depressurization of the pressurizer is started until a time point when pressurization is stopped.

3. The electronic apparatus according to claim 2, wherein the processor is configured to generate the calibration period on a basis of a time difference value of the measurement signal.

4. The electronic apparatus according to claim 3, wherein the processor is configured to determine a period a predetermined time before a maximum value of the time difference value as the calibration period.

5. The electronic apparatus according to claim 4, wherein the time difference value is a first-order differential of time, and a time point when the maximum value arises is set within the depressurization period.

6. The electronic apparatus according to claim 1, comprising a sphygmomanometer comprising the pressurizer and configured to generate the reference blood pressure, wherein
the sphygmomanometer is detachable.

7. The electronic apparatus according to claim 6, wherein the processor is configured to control application of the optical signal, the sphygmomanometer, and the blood pressure acquirer.

8. An electronic apparatus comprising a processor configured to acquire at least a diastolic blood pressure on a basis of the calibration information generated by the electronic apparatus according to claim 1, and a pulse wave corresponding to a received light signal scattered in a body of a subject when an optical signal of a predetermined frequency band is applied thereto.

9. The electronic apparatus according to claim 8, wherein the processor is configured to acquire the diastolic blood pressure on a basis of a first value corresponding to a blood flow rate of the subject in a first period within a period from a first reference time when a value obtained by a first-order differential of the pulse wave with time becomes largest until a second reference time when a next pulse wave rises, and a second value corresponding to a blood resistance of the subject.

10. The electronic apparatus according to claim 9, wherein the processor is configured to acquire a systolic blood pressure further on a basis of a third value corresponding to a blood flow rate of the subject in a second period within a period from a third reference time when the pulse wave rises until a fourth reference time when the pulse wave has a largest peak.

11. The electronic apparatus according to claim 10, wherein the first period is between the first reference time and the fourth reference time, and the second period is between the third reference time and the first reference time.

12. The electronic apparatus according to claim 10, wherein the processor is configured to acquire the third reference time on a basis of a value obtained by dividing a first difference value by a value of the first-order differential providing the largest value, the first difference value being obtained by subtracting a DC component of the pulse wave from a value of the pulse wave at the first reference time.

13. A method comprising:
generating a calibration period corresponding to a pressurization period of a pressurizer for a subject using a received light signal based on scattered light scattered in a body of the subject when an optical signal of a predetermined frequency band is applied thereto;
acquiring a first blood pressure based on the received light signal in the calibration period; and
generating calibration information using a reference blood pressure measured on a basis of pressurization of the pressurizer, and the first blood pressure.
